(19) United States
(12) Patent Application Publication
(10) Pub. No.: US 2026/0241097 A1
(43) Pub. Date: Aug. 20, 2026

(54) Self-Closing Needle and Fluid Loss Prevention Methods

(71) Applicant: Fresenius Medical Care Holdings, Inc., Waltham, MA (US)

(72) Inventors: Kenneth E. BUCKLER, Methuen, MA (US); Michael D. WILLIAMS, Waltham, MA (US)

(73) Assignee: Fresenius Medical Care Holdings, Inc., Waltham, MA (US)

(21) Appl. No.: 19/542,727

(22) Filed: Feb. 18, 2026

Related U.S. Application Data
(60) Provisional application No. 63/760,146, filed on Feb. 19, 2025.

(51) Int. Cl. A61M 5/158

(52) U.S. Cl. CPC A61M 5/158; A61M 2005/1586; A61M 2205/3331; A61M 2209/088

(57) ABSTRACT

A self-closing needle assembly (100) that prevents fluid loss during needle disconnection by incorporating a rigid housing (110, 112), a spring-biased clip (705) with opposing jaws (1207, 1208) to compress a flexible tubing segment (703), and a security tape assembly (102) with adhesive patches (802) and pins (715). The assembly may provide a mechanism that holds the jaws open during use so as to allow unobstructed fluid flow, thereby triggering automatic closure of the tubing segment upon needle withdrawal and ensuring complete shut-off, enhancing patient safety and integrating seamlessly into medical workflows.

### CROSS REFERENCE TO RELATED APPLICATIONS

This Application claims the benefit of US Provisional Application No. 63/760,146 filed February 19, 2025, which is incorporated herein by reference in its entirety.

### BACKGROUND

Over time, medical procedures involving the administration or withdrawal of fluids have come to rely upon assemblies designed to deliver or remove bodily fluids from a patient. These assemblies typically comprise conduits, connectors, and flow-control elements arranged to establish and maintain fluid communication between a patient and a fluid source or collection reservoir. The patient may be a human, but the present disclosure is equally applicable to all animal patients, such as use in veterinary care.

Maintenance of secure fluid pathways during such procedures is important for patient safety and procedural efficacy. In many clinical settings, however, manual clamps and screw-style valves are employed to regulate flow, yet these devices require ongoing attention. Unintentional loosening or disconnection of such mechanisms can go unnoticed until significant fluid loss has occurred, thereby affecting patient hemodynamics.

In particular, extracorporeal blood withdrawal procedures—such as hemodialysis, infusion therapy, and automated blood sampling—are vulnerable to undetected line separation. As a result, inadvertent fluid loss may precipitate hypovolemia or other serious complications that require immediate clinical intervention.

To mitigate these risks, various sensing and alarm systems have been introduced to detect pressure changes or flow interruptions. Such systems often depend on external monitors and audible or visual alerts, requiring operator intervention to secure the line. However, reliance on external power sources or complex electronics can limit compatibility with standard disposable fluid sets and increase overall system cost. Further, power outages may create risks that require non-electronic safety backup options.

Other approaches employ moving external levers which can close fluid flow, but they can suffer from incomplete closure, permitting slow drips or residual flow, and may not reliably seal under varying displacement angles. In some cases, automatic shut-off devices incorporate hinged components that engage only when axial forces exceed a threshold, yet the continuous pressure against the patient can cause discomfort.

Accordingly, clinicians face trade-offs between reliability, ease of use, patient comfort, and cost. As a result, there remains a need for a streamlined fluid line protection system that automatically halts flow upon disconnection without permitting partial occlusion or requiring manual actuation. Such a system is to be fully self-contained and compatible with standard assemblies to facilitate seamless integration into existing workflows, while minimizing external components that could snag or cause patient discomfort.

There is a need for a reliable, binary closure mechanism that delivers either full patency or complete shut-off, thereby eliminating intermittent flow and reducing human error to enhance safety during invasive procedures.

### SUMMARY

The disclosed embodiments address these deficiencies and result in a secure mechanism that completely shuts of flow if the needle is dislodged, do not partially obstruct flow if the needle angle is changed relative to the patient, and provide greater comfort for the patient when compared to existing designs.

According to an aspect of the present disclosure, a self-closing needle assembly comprises a rigid housing, a spring-biased clip disposed within the housing and having opposing jaws configured to compress a flexible tubing segment, the flexible tubing segment fluidly coupling a needle to a tube, and a security tape assembly configured to adhere to a patient and to hold the jaws open during typical operation, wherein withdrawal of the needle from the patient while the security tape assembly remains adhered causes the security tape assembly to release the spring-biased clip and the spring-biased clip automatically compresses the flexible tubing segment to stop fluid flow. The rigid housing provides structural containment and alignment for the spring-biased clip and tubing, ensuring reliable operation; the spring-biased clip delivers automatic closure of the tubing segment upon needle withdrawal, preventing fluid loss; the opposing jaws ensure complete occlusion of the tubing segment for binary shut-off; the flexible tubing segment enables fluid communication between the needle and tube while being susceptible to reliable compression; and the security tape assembly maintains the jaws in an open state during use and triggers closure upon needle dislodgement, thereby preventing inadvertent fluid loss.

According to some embodiments, the spring-biased clip may comprise a tube spring biased toward a closed position, which ensures immediate and forceful closure of the tubing segment when released, thereby preventing partial occlusion and ensuring patient safety. The rigid housing can define openings sized to receive pins of the security tape assembly to hold the jaws open, which enables secure engagement and reliable maintenance of the open state during operation. The spring-biased clip may include an end wall having an aperture through which the flexible tubing segment passes, providing precise positioning and alignment for consistent closure. The flexible tubing segment may include a flattened portion positioned between the jaws for reliable occlusion by the spring-biased clip, which enhances the effectiveness of the closure mechanism and prevents leakage. The self-closing needle assembly may further comprise a support tube segment inserted into an end of the flexible tubing segment to provide structural support at a connection to the needle, which prevents kinking and ensures stable fluid flow during use.

According to an aspect of the present disclosure, a method for preventing fluid loss during needle disconnection comprises providing a needle assembly comprising a rigid housing, a spring-biased clip within the housing, and a flexible tubing segment extending through the spring-biased clip to a needle; providing a security tape assembly; holding jaws of the spring-biased clip open while establishing fluid flow through the flexible tubing segment; adhering the security tape assembly to a patient's skin; and in response to withdrawal of the needle assembly while the security tape assembly remains adhered, permitting the jaws to close and compress the flexible tubing segment to stop fluid flow. Each step ensures that fluid flow is reliably maintained during use and automatically halted upon needle disconnection, thereby preventing inadvertent fluid loss and enhancing patient safety.

According to some embodiments, holding the jaws open may comprise inserting a pair of pins of the security tape assembly through openings in the rigid housing and between the jaws, which ensures secure maintenance of the open state. Adhering the security tape assembly may comprise exposing an adhesive by removing a cover from skin-contact patches on a planar support and pressing the patches to the patient's skin, which provides stable attachment and prevents accidental disengagement. The closure of the flexible tubing segment may be substantially binary such that the flexible tubing segment is substantially fully open while the jaws are held by the pins and substantially fully closed when the pins are withdrawn, which eliminates partial occlusion and ensures reliable shut-off.

According to an aspect of the present disclosure, a security tape assembly for securing a needle placement device comprises a flexible planar support carrying skin-contact adhesive patches configured to adhere the support to a patient's skin, and a pair of pins carried by the support and arranged to extend into a housing of the needle placement device to hold a closure mechanism within the housing in an open state while the device is in place, the pins being withdrawable from the housing upon relative separation between the housing and the adhered support to permit the closure mechanism to compress a flexible tubing segment of the device and halt fluid flow. The flexible planar support provides stable attachment to the patient, the adhesive patches ensure secure adhesion, and the pins enable reliable engagement and disengagement of the closure mechanism for automatic shut-off.

According to some embodiments, the security tape assembly may further comprise pillars extending from the planar support, the pins being mounted on the pillars, which provides structural support and precise positioning for the pins. Wings of the needle hub are secured to a surface with tape that has an adhesive strength. Each adhesive patch of the security tape assembly can have an adhesive strength greater than the adhesive strength of the tape used to secure wings of a needle barrel to a patient, which ensures that the security tape assembly remains adhered during needle withdrawal and reliably triggers closure of the tube spring. Each skin contact patch may include an adhesive covered by a removable cover slip prior to placement, which preserves adhesive integrity until use.

According to some embodiments, the self-closing needle assembly may include a support tube segment inserted into one end of the flexible tubing segment and a tube inserted into the other end of the flexible tubing segment, wherein the support tube segment has a thicker wall thickness than the tube, which provides enhanced structural support and prevents kinking at the connection points, ensuring reliable fluid flow and closure.

According to an aspect of the present disclosure, a method for securing a needle assembly can include providing a needle assembly that comprises a housing (which may be rigid), a spring clip disposed within the housing, and a flexible tubing segment that passes through a hole in an end wall of the spring clip and between a first and a second jaw formed by the top and bottom walls of the spring clip. A security tape assembly that includes a planar support structure and two pillars with pins extending parallel to the structure can be provided. The pillars may have a height based on the size of the housing, so in some cases the pillars may have essentially zero height, so the pins effectively extend out of the planar support structure. The pins can be inserted through openings in the rigid housing and between the jaws of the spring clip to maintain the jaws in an open position. The security tape assembly may be adhered to a patient such that, upon withdrawal of the needle assembly while the security tape remains adhered, the pins retract from between the jaws, permitting the jaws to close and compress the flexible tubing segment due to the spring tension of the spring clip.

According to another aspect of the present disclosure, a self-closing needle may include a rigid housing that may be made out of a polymer, such as a thermoplastic. The rigid housing may be made of two parts – an upper part and a lower part, which when placed together and connected, such as by glue or friction fit, create a hollow cavity inside the housing. The housing has two open ends that fluidly connect to the cavity. Inside the housing is a tube spring which has two opposed jaws urged together by spring force. The opposed jaws may be referred to as an upper jaw and a lower jaw. The two jaws come together at one end of the tube spring, while the opposite end of the tube spring has an end wall, which may be flat or curved. The end wall has a hole formed in it, such that a tube may pass through the hole. The two opposed jaws of the tube spring are held apart from each other by pins that enter the housing at one end. While the pins remain between the jaws, a gap is created between the jaws and the pins, such that the tube that passes through the hole in the tube spring can also pass between the jaws, and exit the housing at the opposite end. This way the tube maintains patency so long as the pins remain between the jaws of the tube spring. The two pins are mechanically attached, or are an integral part of a security tape assembly, which is designed to be adhesively attached to an object which is being cannulated. The tube that passes between the jaws of the tube spring fluidly connects to a cannula which includes a needle mounted to a hub. The hub may have wings and may further include an extension, such as a piece of tubing, between the hub and the housing. When the needle is inserted into a cannulation subject and the security tape assembly is further adhered to the cannulation subject, if the needle assembly is pulled such that the needle withdraws from the cannulation subject, the security tape assembly remains adhered, and thus the pins pull out of the housing and from between the jaws of the tube spring. This causes the tube spring to squeeze the jaws together and stop flow through the tube.

### BRIEF DESCRIPTIONS OF FIGURES

The accompanying drawings, which are incorporated herein and constitute part of this specification, illustrate exemplary embodiments of the disclosure, and, together with the general description given above and the detailed description given below, serve to explain the features of embodiments of the disclosed subject matter. The accompanying drawings have not necessarily been drawn to scale. Where applicable, some features may not be illustrated to assist in the description of underlying features.

FIG. 1A illustrates a perspective view of a self-closing needle assembly, according to embodiments of the disclosed subject matter.

FIG. 1B illustrates a perspective view of a self-closing needle assembly with an extension tube, according to embodiments of the disclosed subject matter.

FIG. 2 illustrates a side-view of a self-closing needle assembly, according to embodiments of the disclosed subject matter.

FIG. 3 illustrates a front view of a self-closing needle assembly, according to embodiments of the disclosed subject matter.

FIG. 4 illustrates a rear view of a self-closing needle assembly, according to embodiments of the disclosed subject matter.

FIG. 5A illustrates a bottom drawing of a self-closing needle assembly, showing a security tape assembly, housing, needle, and medical tubing, according to embodiments of the disclosed subject matter.

FIG. 5B illustrates a bottom view of a self-closing needle assembly with an extension tube, according to embodiments of the disclosed subject matter.

FIG. 6A illustrates a top view of a self-closing needle assembly, according to embodiments of the disclosed subject matter.

FIG. 6B illustrates a top view of a self-closing needle assembly with an extension tube, according to embodiments of the disclosed subject matter.

FIG. 7A illustrates an exploded view of a self-closing needle assembly, according to embodiments of the disclosed subject matter.

FIG. 7B illustrates an exploded view of a self-closing needle assembly with an extension tube, according to embodiments of the disclosed subject matter.

FIG. 8 illustrates a perspective view of a security tape assembly, according to embodiments of the disclosed subject matter.

FIG. 9 illustrates a bottom view of a security tape assembly, according to embodiments of the disclosed subject matter.

FIG. 10A illustrates a perspective view of a lower housing, according to embodiments of the disclosed subject matter.

FIG. 10B illustrates a perspective view of a lower housing, showing groove details for securely holding the tube spring and openings for pin insertion, according to embodiments of the disclosed subject matter.

FIG. 11A illustrates a perspective view of an upper housing assembly, according to embodiments of the disclosed subject matter.

FIG. 11B illustrates a perspective view of an upper housing with a groove designed to securely hold the tube spring, according to embodiments of the disclosed subject matter.

FIG. 12 illustrates a tube spring assembly in the closed position, according to embodiments of the disclosed subject matter.

FIG. 13 illustrates a tube spring in the open position, according to embodiments of the disclosed subject matter.

FIG. 14 illustrates a cross-sectional side view of a self-closing needle assembly, and a security tape assembly with supporting pins holding the jaws open, according to embodiments of the disclosed subject matter.

FIG. 15 illustrates a perspective view of a self-closing needle assembly interacting with the security tape assembly, according to embodiments of the disclosed subject matter.

FIG. 16 illustrates a cross-sectional side view of a self-closing needle assembly, with supporting pins holding the jaws open, according to embodiments of the disclosed subject matter.

FIG. 17 illustrates a cross-sectional side view of a self-closing needle assembly, with supporting pins withdrawn from the jaws, according to embodiments of the disclosed subject matter.

FIG. 18 illustrates a pliable tubing segment, according to embodiments of the disclosed subject matter.

FIG. 19 illustrates a tube spring interacting with a pliable tubing, according to embodiments of the disclosed subject matter.

FIG. 20 illustrates a pliable tubing segment and support tube segment, according to embodiments of the disclosed subject matter.

### DETAILED DESCRIPTION

Medical procedures involving fluid administration or withdrawal, such as hemodialysis, infusion therapy, and automated blood sampling, face significant challenges in maintaining secure and reliable fluid pathways. Existing systems often rely on manual clamps, screw-style valves, or external monitoring devices to regulate and detect fluid flow. These approaches are prone to human error, unintentional disconnection, or delayed response to line separation, leading to risks such as hypovolemia, blood loss, or other serious complications. Additionally, mechanical solutions like spring-loaded clamps or tension-based closures can suffer from incomplete occlusion, permitting slow leaks or residual flow. Many of these designs also rely on external power sources or complex electronics, increasing cost and limiting compatibility with standard disposable fluid sets. Furthermore, some existing mechanisms, such as hinged paddles or axial force-based closures, can cause patient discomfort due to continuous pressure and fail to provide reliable closure under varying displacement angles. These limitations create a significant demand for a streamlined, self-contained solution that ensures complete and automatic fluid shut-off upon needle disconnection.

The disclosed self-closing needle assembly addresses these deficiencies by introducing a fully mechanical closure mechanism that eliminates the need for external power, complex electronics, or manual intervention. The inventive concept integrates a spring-biased clip housed within a rigid structure, a flexible tubing segment, and a security tape assembly. The spring-biased clip features opposing jaws that can compress the flexible tubing segment to achieve a binary closure—either fully open or completely shut—thereby preventing partial occlusion and ensuring reliable fluid flow control, if the security tape assembly is displaced from the spring-biased clip in the housing. The security tape assembly, equipped with adhesive patches and integrated pins, holds the jaws open during standard operation and triggers automatic closure upon needle disconnection. This design ensures that fluid flow is immediately and completely halted if the needle is dislodged, because needle dislodgment will cause the security tape assembly to become spatially displaced from the clip. This prevents inadvertent fluid loss and enhancing patient safety. The rigid housing provides structural containment and alignment for the spring-biased clip, while the flexible tubing segment may include a flattened portion to ensure effective occlusion without compromising structural integrity of the tubing.

By eliminating reliance on external power sources, reducing the risk of human error, and ensuring compatibility with standard medical assemblies, the disclosed technology offers a cost-effective, reliable, and patient-friendly solution. The binary closure mechanism, combined with the innovative use of a security tape assembly, ensures seamless integration into existing workflows while minimizing patient discomfort and procedural complexity. This streamlined approach significantly improves safety and efficiency in medical procedures, addressing the longstanding challenges associated with fluid line disconnection.

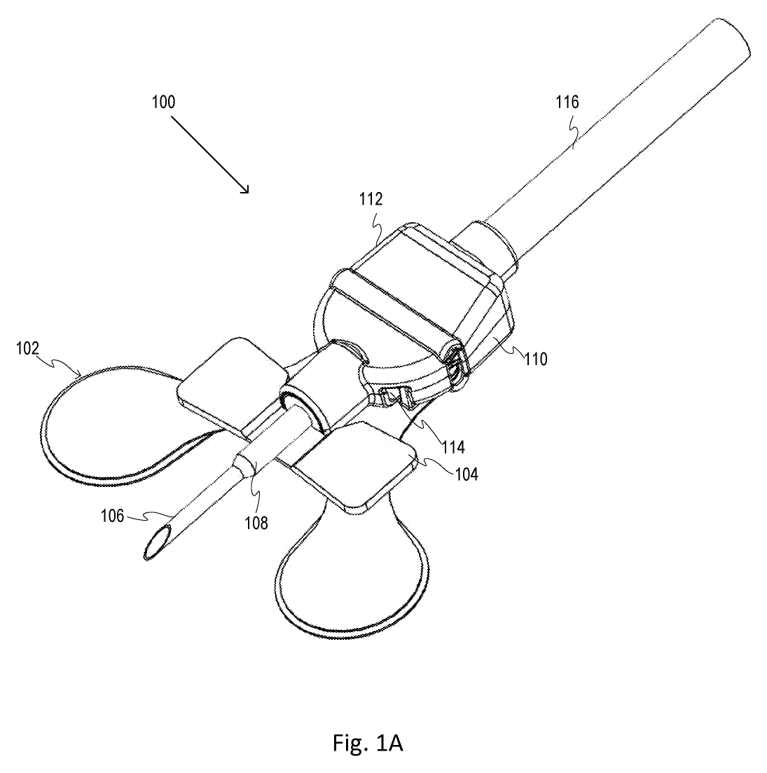

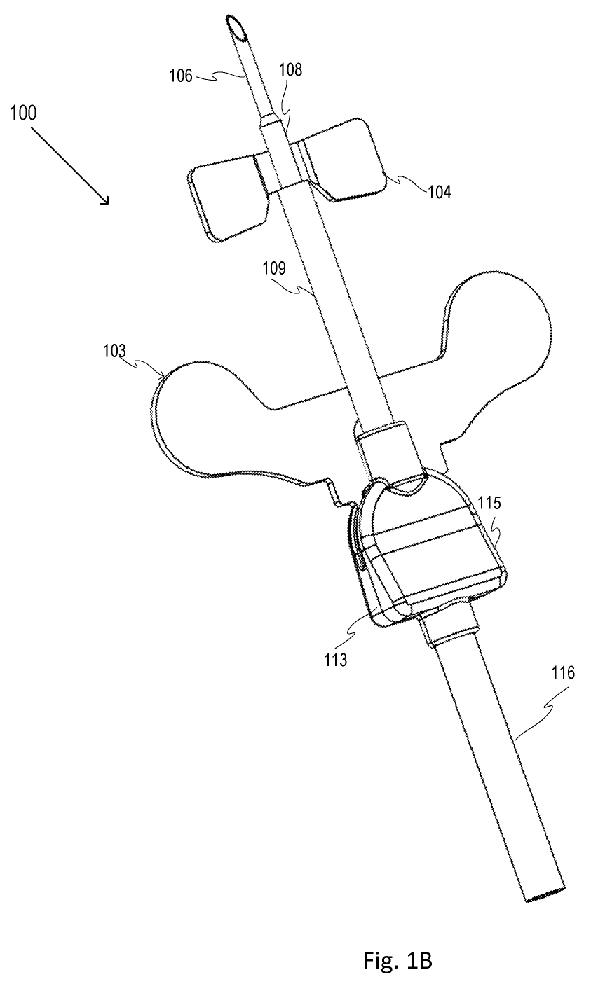

Referring now to FIG. 1A, a perspective view of a self-closing needle assembly 100 is shown, which is designed to prevent fluid loss during needle disconnection. The assembly 100 integrates several components that contribute to secure and reliable operation during medical procedures.

A security tape assembly 102 is provided, which is configured to adhere to a patient’s skin and maintain a specific spatial relationship between the needle assembly 100 and the security tape assembly 102.

A needle 106 is shown protruding from a needle barrel 108, which serves as the primary housing for the needle 106. In the present embodiment, the needle barrel 108 is equipped with wings 104 that extend laterally on either side. These wings 104 are typically used to secure the needle assembly 100 to a surface into which the needle 106 is inserted, using medical tape, providing additional stability, and preventing unintended movement of the needle. Under normal use, when the needle does not become dislodged, the flow through the needle assembly remains open. It will be apparent that if the needle 106 is withdrawn from an object in which it was inserted (e.g., the medical tape is not strong enough to keep the needle 106 inserted), this will cause the whole housing of the needle assembly to also move in the same direction as the needle. This will cause spatial relationship between the security tape assembly 102 and the housing to change, because the security tape assembly 102 is separately adhered to the surface in which the needle is inserted.

The needle barrel 108 may be connected directly to a rigid housing, which is composed of an upper housing 112 and a lower housing 110, or it may be connected to the rigid housing through extension tube 109, which is illustrated in FIG. 1B. These two housing components enclose an internal spring-biased clip (not visible in this figure) that facilitates the automatic closure of the tubing upon needle disconnection – because needle disconnection will cause the security tape assembly 102 to become spatially displaced from the housing. The housing is designed to provide structural containment and alignment for the internal components, ensuring reliable operation under varying procedural conditions.

The housing further includes openings 114, which are configured to receive pins 715 from the security tape assembly 102. These pins hold the internal spring-biased clip in an open position during typical operation, thereby allowing fluid to flow through a tape that passes between the open jaws of the tube spring 705. Upon needle disconnection, the pins 715 are withdrawn and, as a result, the spring-biased clip 705 compresses the tubing to halt fluid flow.

Medical tubing 116 extends from the rear end of the housing, providing a fluid pathway between the needle 106 and an external fluid source or collection reservoir. The design of the tubing 116 allows for maintaining patency during use while being susceptible to reliable compression by the spring-biased clip, thereby ensuring a thorough shut-off when required.

Referring now to FIG. 1B, a self-closing needle assembly 100 is shown, with particular emphasis on the extension tube 109 and the modified shape of the security tape assembly 103. The extension tube 109 is positioned between the needle barrel 108 and the rigid housing, which comprises an upper housing 115 and a lower housing 113. The extension tube 109 may be flexible or semi-rigid, providing a balance between flexibility and structural integrity. This configuration allows the extension tube 109 to maintain its general shape and avoid kinking, thereby ensuring reliable fluid flow and reducing the risk of occlusion. It should be readily apparent that in this configuration, as well as in the one of FIG. 1A, if tube 116 is pulled with sufficient force, needle 106 may be withdrawn from the surface into which it was inserted. But the security tape assembly 102 or 103 will remain adhered, and the movement of the housing (made of upper and lower parts shown) will cause pins 715 to be withdrawn from the housing, and consequently from the jaws of the tube spring 705, permitting the tube spring 705 to close and pinch the tube passing through its jaws.

The extension tube 109 may have a length that is related to the length of the needle 106. In an embodiment, the extension tube 109 is 1 to 2 times the length of the needle 106. The minimum length of extension tube 109 may be selected to be the same as the width of the medical tape that is used to attach wings 104 to the patient. As can be appreciated, the wings 104 will be covered by medical tape, and this tape will generally be centered over the wings 104, so that about one half of the tape’s width will extend over the extension tube 109. Thus, making the extension tube 109 at least as wide as the tape, will generally ensure that the tape does not interfere with the security tape assembly 103. It should also be appreciated that the length of extension tube 109 should not exceed a certain length to make the overall device comfortable for use. In an exemplary embodiment, the length of extension tube 109 is between 3.5 cm and 4 cm.

The provision of the extension tube 109 offers an advantage in terms of patient comfort. By allowing the needle 106 to enter the patient at a lower angle, the extension tube 109 reduces the likelihood of discomfort that may arise from high-angle needle insertion. This feature is particularly beneficial in clinical settings where patient mobility or anatomical constraints require a more flexible approach to needle placement.

Additionally, the security tape assembly 103 in FIG. 1B exhibits a modified shape compared to the equivalent security tape assembly 102 shown in FIG. 1A. The altered geometry of the security tape assembly 103 may enhance the ability of the tape to conform to the patient’s skin and provide improved adhesion and stability during use. The integration of these features within the self-closing needle assembly 100 contributes to a more comfortable and reliable solution for preventing fluid loss during needle disconnection.

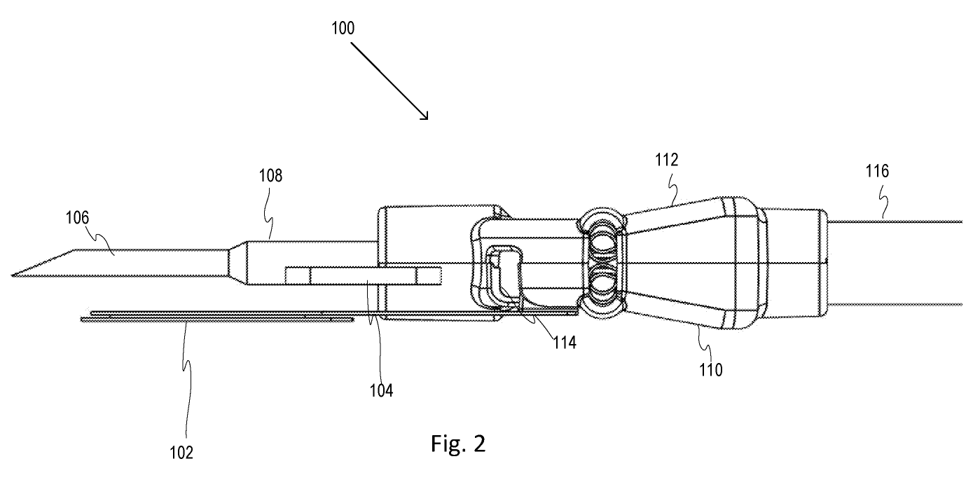

Referring now to FIG. 2, a side view of a self-closing needle assembly 100 is shown, illustrating the main components and their arrangement to achieve secure and reliable operation during medical procedures. The assembly 100 includes a needle 106 which is connected to a needle barrel 108. The needle barrel 108 serves as the primary housing for the needle 106 and provides structural support. Extending laterally from the needle barrel 108 are wings 104, which are configured to stabilize the assembly when secured to a patient’s skin using medical tape, thereby enhancing overall stability, and preventing unintended movement during use.

In the present embodiment, a security tape assembly 102 is positioned beneath the needle barrel 108. The security tape assembly 102 is configured to adhere to the patient’s skin, ensuring that the needle assembly 100 remains securely in place during operation and to trigger the automatic closure mechanism upon needle disconnection. Accordingly, the security tape assembly 102 cooperates with internal components to effectuate rapid shut-off of fluid flow when the needle 106 is withdrawn.

The needle barrel 108 is connected to a rigid housing composed of an upper housing 112 and a lower housing 110. These housing components enclose an internal spring-biased clip that facilitates automatic closure of the tubing upon needle disconnect. The upper housing 112 and lower housing 110 are designed to provide structural containment and alignment for the internal components, ensuring reliable operation under varying procedural conditions. The design of the housings 110 and 112 allows them to be molded in a two-part mold and assembled to form a variety of shapes without compromising performance.

Furthermore, the housing includes openings 114, which are configured to receive pins 715 from the security tape assembly 102. The pins 715 hold the internal spring-biased clip in an open position during typical operation, permitting fluid flow through the assembly. Upon needle disconnection, the pins 715 are withdrawn and, as a result, the spring-biased clip compresses the medical tubing 116, halting fluid flow. The medical tubing 116 extends from the rear end of the housing, providing a fluid pathway between the needle 106 and an external fluid source or collection reservoir. In this manner, the medical tubing 116 is maintained in a patent state during use yet is susceptible to reliable compression by the spring-biased clip to ensure complete shut-off when required. In embodiments, the self-closing needle assembly 100 may be used on the venous blood line of a hemodialysis or hemofiltration system, as the venous line conveys a large volume of blood back to the patient. If that line were to become disconnected and flow were to continue, a significant amount of blood could be lost.

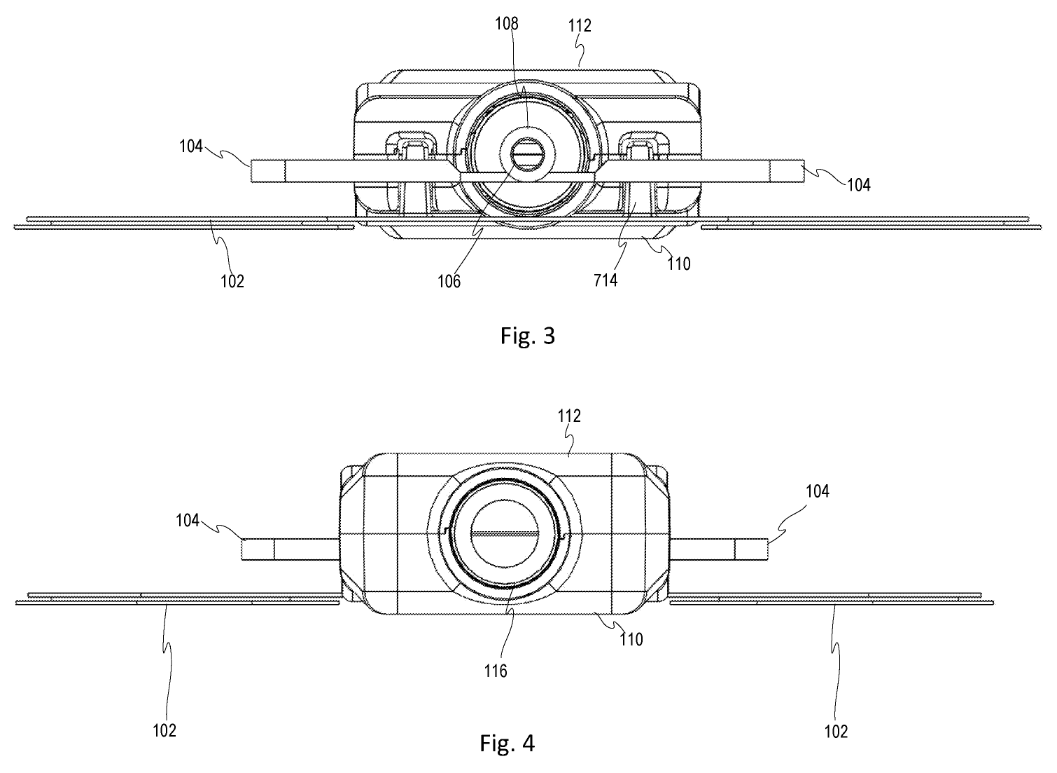

Referring now to FIG. 3, a front view of the self-closing needle assembly 100 is presented, illustrating the spatial arrangement of the primary components within the assembly. In the present embodiment, the needle barrel 108 is centrally positioned and serves as the main structural component for supporting the needle 106. Extending laterally from the needle barrel 108 are a first pair of wings 104, which provide stability and facilitate secure attachment of the assembly to the patient using medical tape. These wings 104 are symmetrically disposed on opposite sides of the needle barrel 108, thereby ensuring balanced support during use.

The needle barrel 108 is connected to a rigid housing composed of an upper housing 112 and a lower housing 110. Accordingly, the upper housing 112 and lower housing 110 together enclose an internal spring-biased clip – referred to as tube spring 705 with reference to other FIGS. - that facilitates automatic closure of the tubing upon needle disconnection. The design of the housing components provides structural containment and precise alignment for the internal elements, supporting dependable functionality under varying procedural conditions.

Beneath the housing, the security tape assembly 102 is visible. Although the security tape assembly 102 is illustrated as flat, the assembly is actually flexible enough to adopt a shape that matches the shape of the surface into which the needle 106 is inserted. For example, this could be the arm of a human patient. The security tape assembly 102 may include pillars 714 that support pins 715 (not shown in this figure) used to hold the internal spring-biased clip in an open position during typical operation. Moreover, the security tape assembly 102 is configured to adhere to the patient’s skin, ensuring that the needle assembly 100 remains securely in place during use. The pillars 714 are positioned to align with corresponding openings 114 in the housing, thereby enabling the pins 715 to engage with the internal spring-biased clip.

In embodiments where an extension tube 109 is interposed between the needle barrel 108 and the housing, the elevation of the wings 104 will generally match the elevation level of the security tape assembly 102. This is achieved due to the flexibility of the extension tube 109, which allows the wings 104 to be positioned at a similar height as the security tape assembly 102, thereby improving comfort and stability during use.

This front view emphasizes the compact and integrated design of the self-closing needle assembly 100, which merges mechanical reliability with user-friendly features to improve safety and operational effectiveness in medical procedures. The arrangement of the components demonstrates that the assembly maintains stability and functionality during operation, while the security tape assembly 102 adds an extra measure of safety by activating the automatic closure mechanism when the needle 106 is disconnected.

FIG. 4 shows a rear view of the self-closing needle assembly 100, illustrating the arrangement of components from the perspective opposite to FIG. 3. The security tape assembly 102, wings 104, upper housing 112, and lower housing 110 are visible in this view; these elements have been described above in connection with FIG. 3.

In this embodiment, medical tubing 116 is shown extending from the rear of the housing, providing a fluid pathway for connection to an external fluid source or collection reservoir. The positioning of medical tubing 116 at the rear of the assembly facilitates reliable fluid communication and supports the binary closure mechanism of the internal spring-biased clip. The housing formed by the upper housing 112 and lower housing 110 ensures structural containment and alignment for the medical tubing 116, enabling consistent operation of the closure mechanism upon needle disconnection.

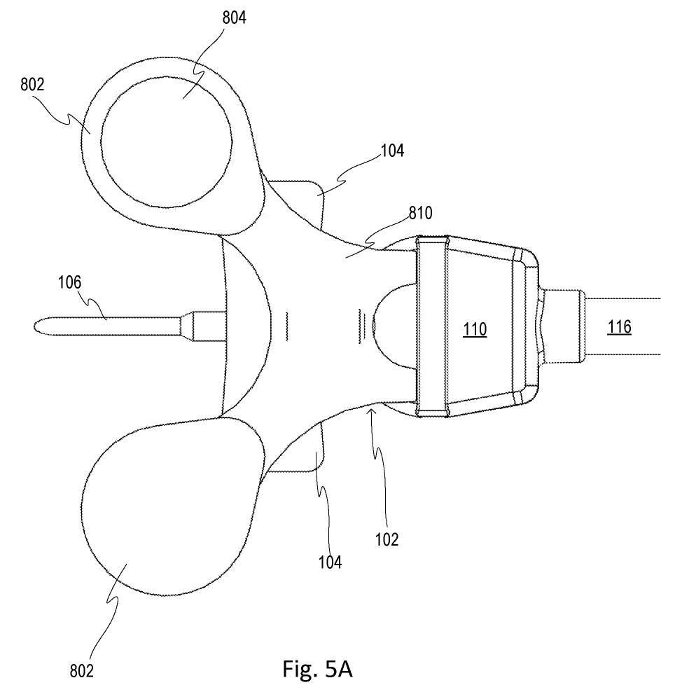

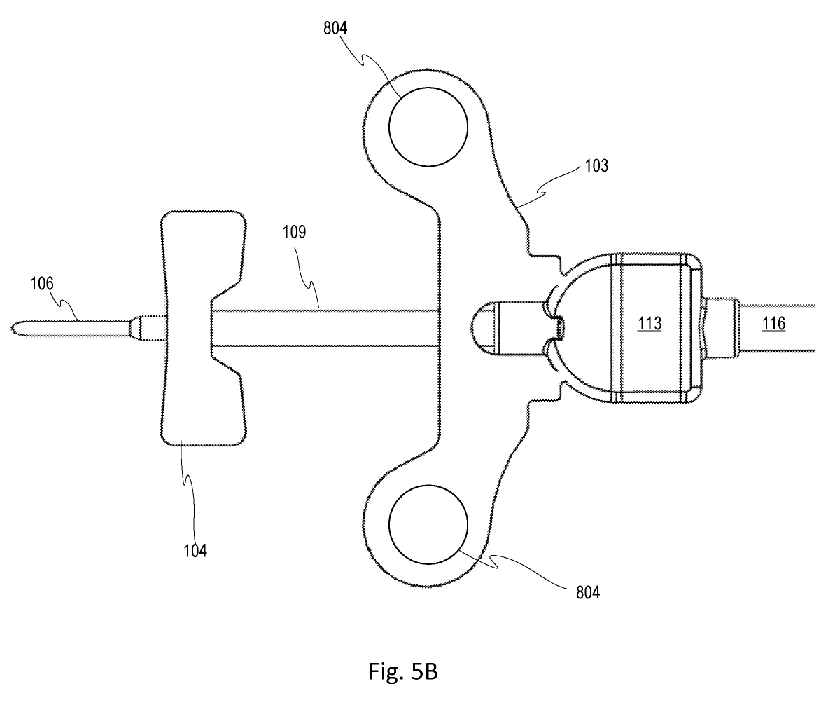

FIG. 5A shows a bottom view of a self-closing needle assembly 100 without an extension tube 109. The security tape assembly 102 is depicted with the planar support structure 810 extending laterally, providing a stable base for secure adhesion to the patient’s skin. Skin contact patches 802 and adhesive portions 804 are positioned at the ends of the planar support structure 810 to ensure reliable attachment during use. The wings 104 and needle 106 are centrally positioned, facilitating fluid access and stabilization. The lower housing 110 encloses the internal spring-biased clip and provides structural containment for the internal components. Medical tubing 116 extends from the lower housing 110, establishing a fluid pathway between the needle 106 and an external fluid source or collection reservoir.

FIG. 5B shows a bottom view of a self-closing needle assembly including the extension tube 109. The security tape assembly 103, which features a modified shape, is shown with adhesive portions 804 at the ends to ensure secure attachment to the patient’s skin. The wings 104 and needle 106 are centrally positioned, while the extension tube 109 is interposed between the needle 106 and the lower housing 113. This configuration allows for greater flexibility and improved patient comfort by reducing the likelihood of discomfort from high-angle needle insertion. The lower housing 113 encloses the internal spring-biased clip and provides structural containment. Medical tubing 116 extends from the lower housing 113, establishing a fluid pathway as previously described. The arrangement in FIG. 5B demonstrates the integration of the extension tube 109, which enhances the adaptability and comfort of the self-closing needle assembly during use.

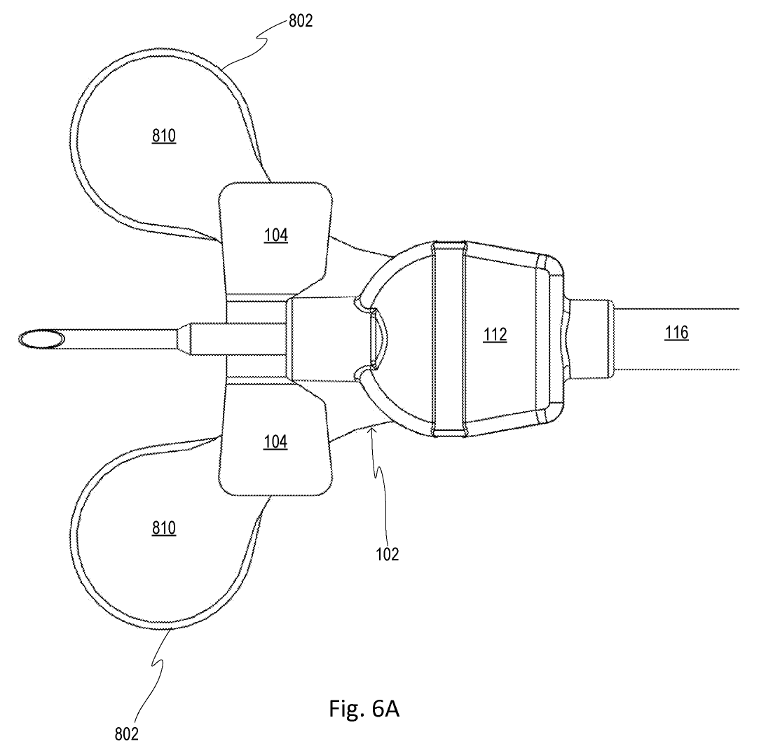

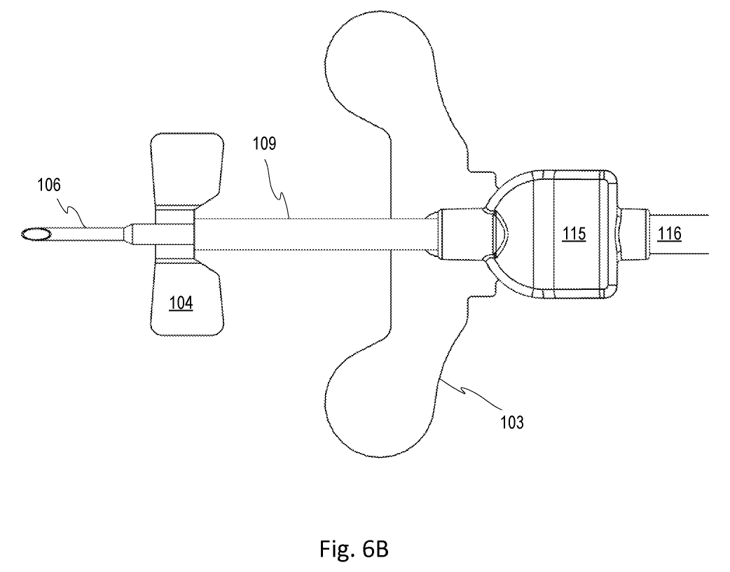

Referring now to FIG. 6A, a top view of a self-closing needle assembly 100 is shown, illustrating the spatial relationship and functional arrangement of the primary components during use. In this example, the security tape assembly 102 is positioned beneath and overlaps under the wings 104 of the needle barrel 108. When the needle 106 is in clinical use, medical tape is placed over the wings 104 and also over a portion of the security tape assembly 102. This overlapping configuration enhances stability and helps ensure that the security tape assembly 102 remains adhered to the patient’s skin even if the wings 104 are inadvertently loosened or displaced.

The needle 106 is centrally located and protrudes from the needle barrel 108, which serves as the primary structural support for the cannula assembly. The needle barrel 108 includes wings 104 extending laterally on opposite sides, providing broad contact areas for secure taping and improved stabilization. The security tape assembly 102, in this top view, is situated such that portions of its planar support 810 lie beneath the wings 104. The skin-contact patches 802 of the security tape assembly 102 are configured to be adhered to the patient’s skin with adhesive portions 804, thereby securing the assembly and contributing to reliable activation of the automatic closure mechanism when needed.

The upper housing 112 forms the top component of a rigid housing that encloses an internal spring-biased clip configured to compress a flexible tubing segment upon release. The upper housing 112 is shaped to provide structural containment and alignment for the internal elements, including the tube spring and the pliable tubing segment, facilitating reproducible operation of the binary closure mechanism. Medical tubing 116 extends from the rear of the housing, establishing fluid communication between the needle 106 and an external system such as a fluid source or collection reservoir. During normal operation, the internal clip is held open by the pins of the security tape assembly 102; upon needle dislodgement while the security tape assembly 102 remains adhered, the pins 715 withdraw and the tube spring 705 closes the fluid path from tubing 116 to stop fluid flow.

In use, the overlap between the wings 104 and the security tape assembly 102 supports a dual anchoring approach. Medical tape applied across the wings 104 simultaneously captures and engages a portion of the security tape assembly 102. This configuration helps maintain the positional integrity of the assembly on the patient’s skin, reduces the likelihood of accidental displacement, and promotes reliable triggering of the automatic shut-off if the needle 106 is withdrawn. The arrangement visible in FIG. 6A demonstrates how the needle 106, needle barrel 108, wings 104, security tape assembly 102, upper housing 112, and medical tubing 116 cooperate to ensure stable placement, consistent fluid patency during use, and immediate, complete occlusion upon disconnection.

Referring now to FIG. 6B, a top view of a medical device assembly is shown that includes a needle 106, wings 104, an extension tube 109, a security tape assembly 103 of modified shape, a upper housing 115, and medical tubing 116. In this configuration, the extension tube 109 is interposed between the needle barrel and the upper housing 115. The extension tube 109 positions the security tape assembly 103 at a location spaced away from the wings 104, such that when the wings 104 are taped down with medical tape, the medical tape does not contact or capture any portion of the security tape assembly 103. Instead, the security tape assembly 103 is independently adhered to the patient via its skin-contact adhesive regions and remains separate from the medical tape used to secure the wings 104.

This spatial separation offers functional benefits during clinical use. Because the security tape assembly 103 is not held in place by the medical tape that secures the wings 104, any loss of adhesion of the security tape assembly 103 to the patient is readily detectable because the security tape assembly 103 will not remain inadvertently retained by the wing tape. If a loss of adhesion between the security tape assembly 103 and the target surface (e.g., patient skin) is observed, the patient’s skin can be cleaned and a new needle assembly can be used, thereby maintaining reliable operation of the automatic shut-off mechanism. The upper housing 115 encloses the internal spring-biased closure mechanism and aligns with the medical tubing 116 extending distally to an external system. Thus, the extension tube 109 provides strain relief and positional flexibility, while maintaining fluid communication between the needle 106 and the housing 115, and simultaneously enabling the independent placement and adhesion of the security tape assembly 103 for improved safety and workflow.

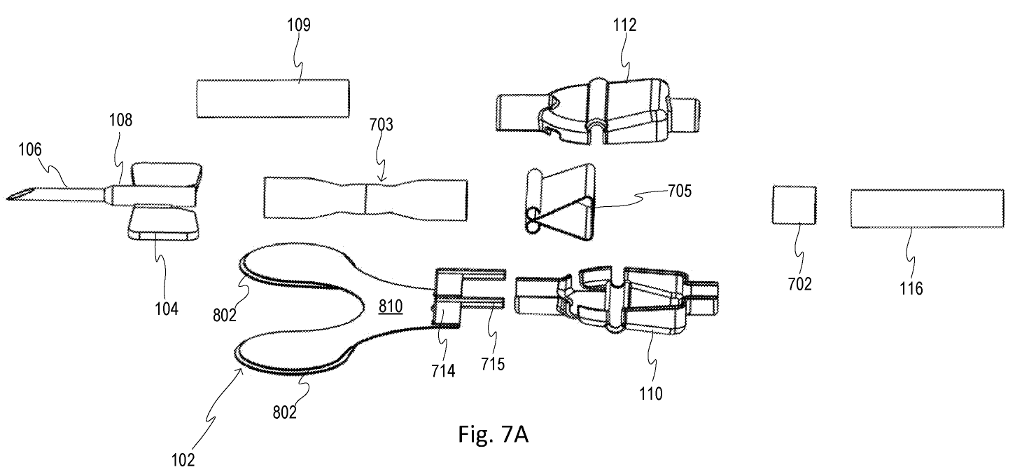

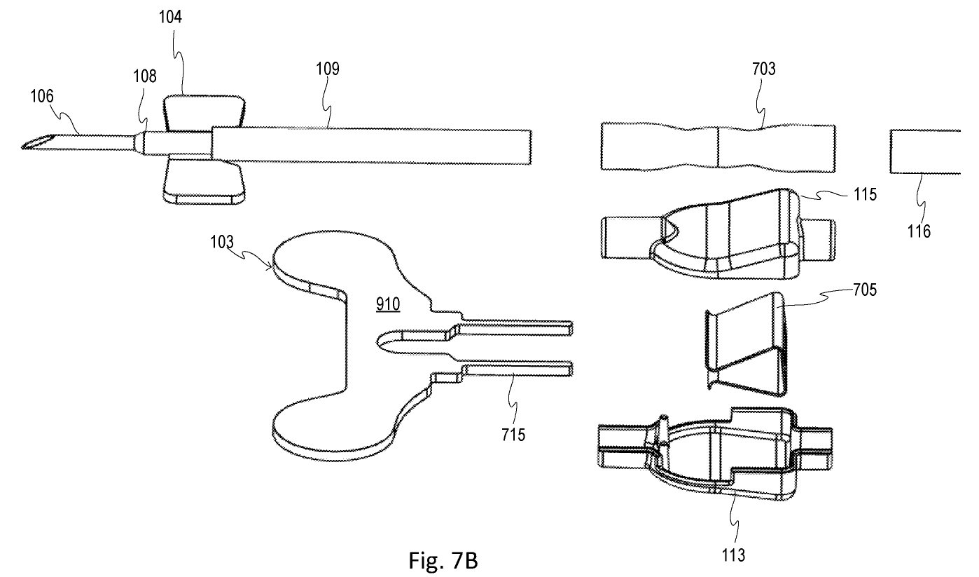

Referring now to FIG. 7A, an exploded view of a self-closing needle assembly 100 is shown, corresponding to the configuration of FIG. 1A and further including an extension tube 109. From proximal to distal, the cannula assembly includes a needle 106 mounted to a needle barrel 108 having laterally extending wings 104. The extension tube 109 can be interposed between the needle barrel 108 and the housing to provide positional flexibility and strain relief while preserving fluid communication, or it may be omitted in certain situations.

A pliable tubing segment 703 is configured with rounded end portions and a molded flattened portion positioned to interact with an internal tube spring 705. The tube spring 705 is biased toward a closed position and includes an end wall with a hole and opposing jaws defining a jaw opening. The pliable tubing segment 703 passes through the jaw opening and the hole to align the flattened portion between the jaws for reliable occlusion when the spring closes. In some embodiments, the pliable tubing segment 703 may be omitted, and tubing segment 116 itself may take place of the pliable tubing segment 703.

The security tape assembly includes a planar support 810 carrying skin contact patches 802 and pillars 714 that support pins 715. The pins 715 are sized and positioned to enter the housing and hold the jaws of the tube spring 705 open during normal operation. The housing is formed from a lower housing 110 and an upper housing 112 that, when assembled, define an internal cavity to receive the tube spring 705 and guide the pliable tubing segment 703 or the tubing segment 116. Openings 114 in the housing align with the pins 715 for insertion and withdrawal.

A support tube segment 702 may be inserted into one rounded end of the pliable tubing segment 703 to reinforce the connection adjacent to the needle barrel 108 and resist kinking. Medical tubing 116 is connected to the opposite end of the pliable tubing segment 703 to establish fluid communication with external systems. In assembly, the components shown in FIG. 7A cooperate such that the pins 715 hold the tube spring 705 open to maintain patency of the pliable tubing segment 703; upon separation of the housing from the adhered security tape assembly 102, the pins 715 withdraw and the tube spring 705 closes the jaws to compress the flattened portion of the pliable tubing segment 703 and stop fluid flow. The spring 705 has sufficient spring force to completely occlude pliable tubing segment 703.

Referring now to FIG. 7B, an exploded view of a self-closing needle assembly 100 is shown corresponding to the configuration of FIG. 1B, with the addition of an extension tube 109 attached to the needle barrel 108. From proximal to distal, the cannula assembly includes a needle 106 mounted to a needle barrel 108 having laterally extending wings 104. The extension tube 109 is secured to the needle barrel 108 and provides positional flexibility and strain relief while maintaining fluid communication from the cannula to the housing.

A security tape assembly 103 of modified shape is shown with a planar support structure 910 configured to conform to patient anatomy. The planar support structure 910 carries two pins 715 that extend parallel to the support. The pins 715 are sized and positioned to enter the housing and hold open the jaws of a tube spring 705 during normal operation.

The housing is formed from an upper housing 115 and a lower housing 113 which, when assembled, define an internal cavity that receives the tube spring 705 and guides a pliable tubing segment 703. The tube spring 705 includes an end wall with a hole and opposing jaws that define a jaw opening. The pliable tubing segment 703 passes through the jaw opening and the hole so that a flattened portion of the tubing segment is positioned between the jaws for reliable occlusion when the spring closes. Medical tubing 116 connects distally to the pliable tubing segment 703 to establish fluid communication with external systems. In some scenarios, the pliable tubing segment 703 may be omitted, such that extension tube 109 and tubing segment 116 are one piece of tubing passing through the housing and through the tube spring 705, which simplifies manufacturing and reduces overall cost of the self-closing needle assembly.

In assembly, the pins 715 of the security tape assembly 103 are inserted through openings in the housing to hold the jaws of the tube spring 705 open, maintaining patency of the pliable tubing segment 703. Upon separation of the housing from the adhered security tape assembly 103, the pins 715 withdraw and the tube spring 705 closes the jaws to compress the flattened portion of the pliable tubing segment 703 and stop fluid flow. The incorporation of the extension tube 109 attached to the needle barrel 108 allows the cannula to be positioned at a lower entry angle while preserving the functional relationship among the needle 106, wings 104, modified security tape assembly 103 with planar support structure 910 and pins 715, upper housing 115, lower housing 113, tube spring 705, pliable tubing segment 703, and medical tubing 116.

In the embodiment of FIG. 7B, the security tape assembly 103 employs a planar support structure 910 that is shaped to conform around the cannula and housing while presenting broad, skin-contact regions for adhesion. The planar support structure 910 carries two pins 715 that are formed or mounted to extend generally parallel to the plane of the support. Unlike embodiments that utilize pillars 714 to elevate the pins, the present configuration omits pillars altogether. The pins 715 are positioned at the perimeter of the planar support structure 910 so that, when the assembly is placed adjacent the housing, the pins 715 align with openings in the upper housing 115 and lower housing 113 and slide directly into the tube spring’s jaw opening to hold the jaws open during normal operation.

By eliminating pillars, the planar support structure 910 can be produced as a substantially flat component from an appropriately thick, flexible medical-grade plastic. The outline of the planar support structure 910 and the integrated or attached pins 715 can be fabricated using stamping, die-cutting, laser cutting, or similar sheet-conversion processes. This approach simplifies manufacturing, reduces part count, and enables tight dimensional control for consistent pin alignment with the housing openings. The flat profile also improves conformability to patient anatomy and reduces bulk under medical dressings.

The planar support structure 910 includes skin-contact adhesive regions configured to adhere the assembly 103 directly to the patient’s skin independently of the medical tape that secures wings 104. Because the security tape assembly 103 is not captured by wing tape, any loss of adhesion is readily detectable and prompts clinical remediation, such as cleaning the patient’s skin and replacing the needle assembly. During use, the pins 715 maintain the tube spring 705 in an open condition; upon separation of the housing from the adhered planar support structure 910, the pins 715 withdraw and the tube spring 705 closes to compress the pliable tubing segment 703 and halt fluid flow.

Upon removal of the needle 106 from the patient at the conclusion of treatment, the security tape assembly 103 remains adhered to the patient’s skin. In this context, the pins 715 serve as convenient leverage points for a medical practitioner to grasp and lift upward on the planar support structure 910, thereby detaching the security tape assembly 103 from the patient in a controlled manner. This removal procedure provides a clear tactile feature to assist peel-off while minimizing shear on the skin and maintaining predictable disengagement from the housing.

An example of the manufacturing process for making the simplified self-closing assembly follows. A conventional needle 106 with hub 108 and wings 104 has a piece of tubing pre-attached, and is supplied as a bulk disposable part. The tube spring 705 is held open with a jig or another tool, to an opening greater than the height of the pins 715. This larger opening of the jaws makes it easy to pass the tube through the jaws, as compared to the opening size created by pins 715. While the tube spring 705 is held in this orientation, the tube segment extending from the hub 108 is inserted between the jaws of the tube spring 705 and out of the back opening 1202 of the tube spring 705. The tube is slid into appropriate position such that the portion between the hub 108 and the jaws of the tube spring 705 are of an appropriate length, corresponding to what is described for extension tube 109 in this disclosure. At this point, pins 715 of the security tape assembly are inserted into the open jaws of the tube spring 705, and the jig or tool that was holding the jaws open is removed. Next, the upper housing 115 and the lower housing 113 are brought together to enclose the tube spring 705 and the tube passing through it. It will be apparent that the design of openings on the lower housing 113 makes this possible and easy to align. The upper and lower housing may be bonded together via adhesive, heat, plastic welding, laser welding, or other standard mechanical joining methods. Since the opening between the jaws of the tube spring 705 is now determined by the thickness of pins 715, the opening of the jaws is small enough to resist sliding of the tube through the jaws, but still allows unobstructed flow of fluid through the tube. At this stage, a finished self-closing needle assembly 101, as shown completed in FIG. 1B is completed. The end of tubing segment 116 may then be furnished with a luer connector, or a similar connector used in the medical field. The completed product may then be sterilized and packaged.

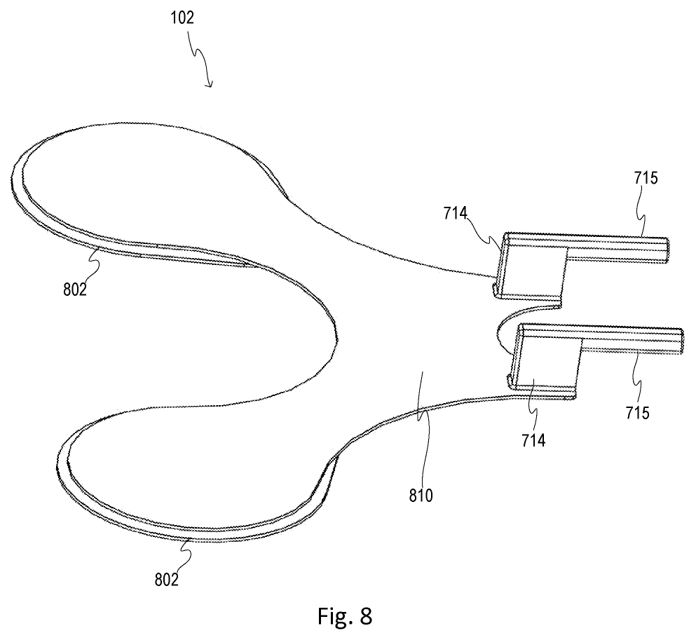

Referring now to FIG. 8, a perspective view the security tape assembly 102 is shown. The security tape assembly 102 includes a planar support structure 810 formed from a flexible, medical-grade material and shaped to present broad contact regions suitable for adherence to a patient’s skin. At two opposed ends of the planar support structure 810 are skin contact patches 802, each sized and positioned to provide stable adhesion while allowing clearance for needle placement and taping of the cannula wings. The skin contact patches 802 carry adhesive portions configured for secure attachment to the patient during clinical use.

Proximal to the housing-facing side of the planar support structure 810 are pillars 714 that extend generally perpendicular from the plane of the support. Each pillar 714 mounts a pin 715 that extends parallel to the planar support structure 810. The pins 715 are dimensioned and spaced to align with openings 114 in the rigid housing so that, when inserted, the pins 715 enter the jaw opening of the tube spring and hold the spring’s opposing jaws in an open position during normal operation. In this manner, the pins 715 maintain patency of the pliable tubing segment 703 while the assembly is in place. Upon relative separation of the housing from the adhered security tape assembly 102, the pins 715 withdraw from the jaw opening, permitting the tube spring 705 to close and compress the pliable tubing segment 703 to stop fluid flow.

In use, after the needle 106 has been withdrawn from the patient at the conclusion of treatment, the security tape assembly 102 remains adhered to the patient’s skin. The pins 715 provide convenient leverage points that a medical practitioner can grasp and lift to peel the planar support structure 810 away from the skin in a controlled manner. This feature facilitates removal while minimizing shear forces on the patient’s skin and ensures predictable disengagement from the housing.

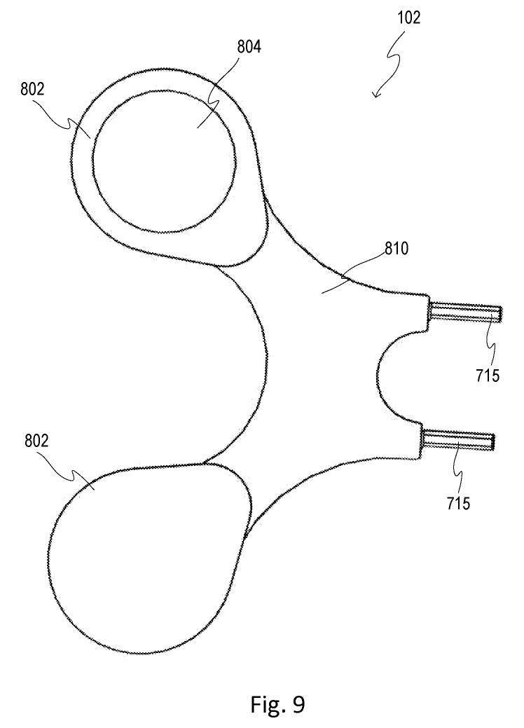

Referring now to FIG. 9, a bottom view of the security tape assembly 102 is shown. In this view, the planar support structure 810 extends laterally to present broad contact regions configured for adhesion to a patient’s skin. Two skin contact patches 802 are visible on opposed ends of the planar support structure 810. Each skin contact patch 802 carries an adhesive portion 804 (even though only one adhesive portion 804 is shown here for clarity) arranged to provide secure attachment during clinical use use while maintaining clearance for placement of the cannula and taping of wings.

The pins 715 are shown projecting from the housing-facing side of the planar support structure 810. In the assembled device, the pins 715 are aligned with openings in the rigid housing and are inserted to engage the jaw opening of the internal tube spring, thereby holding the jaws open to maintain patency of the pliable tubing segment during normal operation. When the housing separates from the adhered security tape assembly 102—such as when the needle assembly is withdrawn—the pins 715 retract from the tube spring 705, allowing the spring to close and occlude the tubing.

This bottom view emphasizes the functional arrangement of the adhesive portions 804 on the skin contact patches 802, the geometry of the planar support structure 810 that supports stable adhesion and proper alignment, and the positioning of the pins 715 that enable engagement with the housing and reliable activation of the automatic shut-off mechanism upon disconnection.

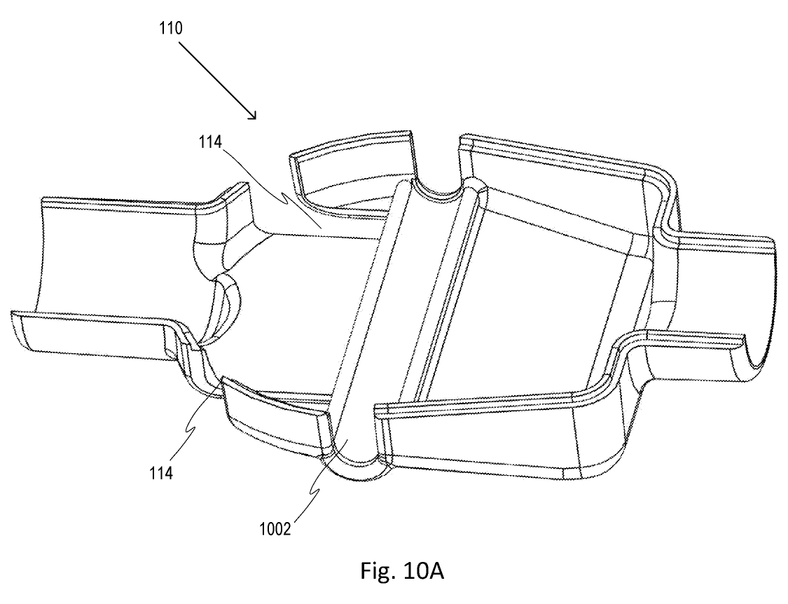

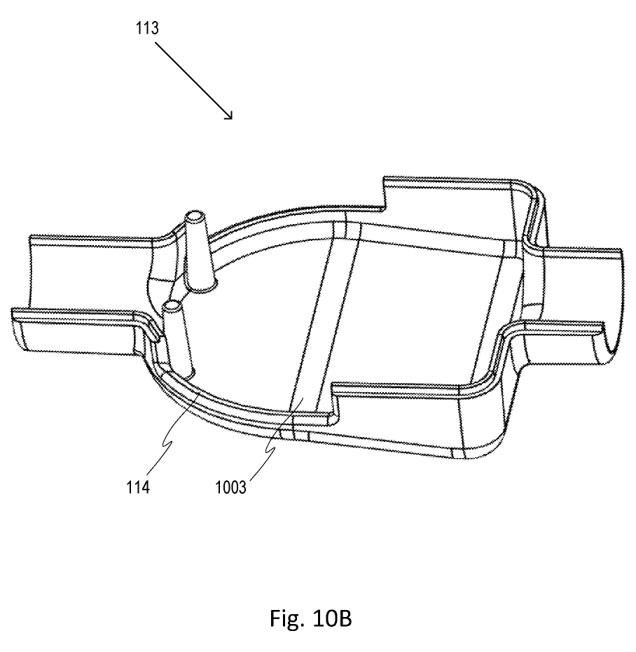

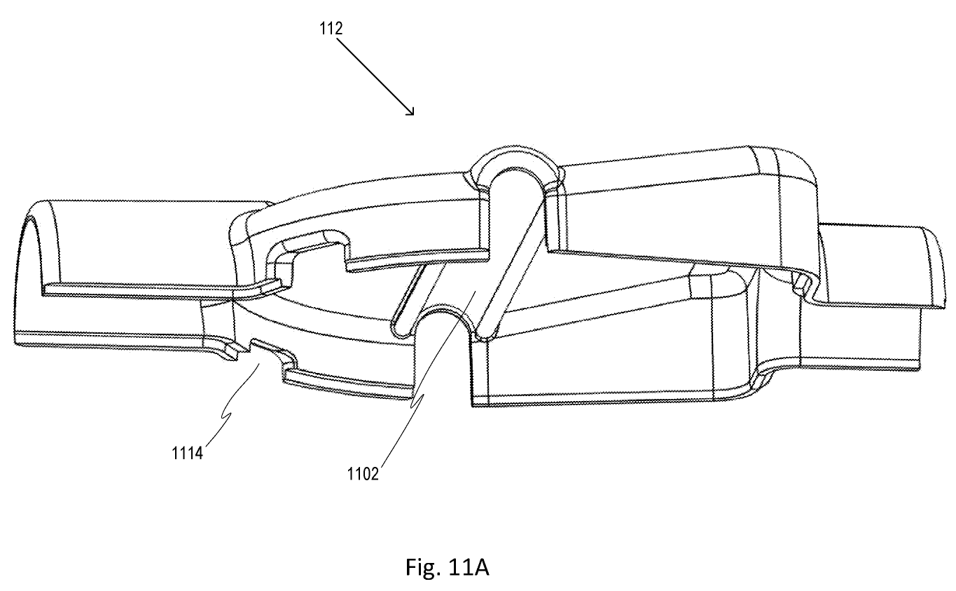

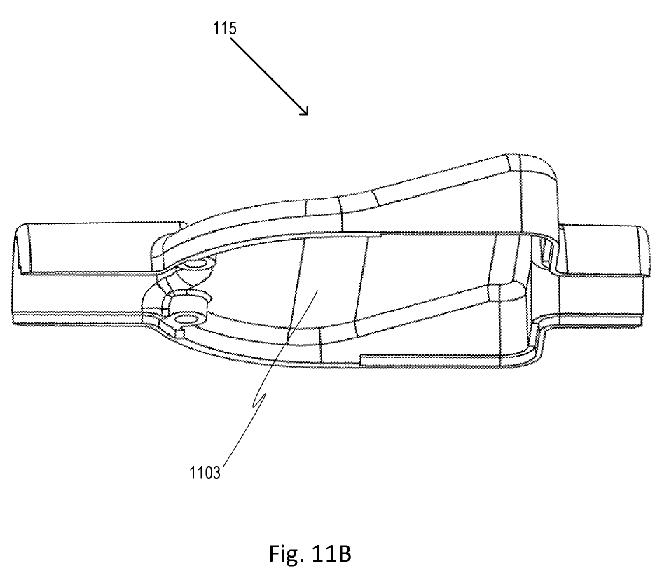

Referring now to FIGS. 10A and 11A, the lower housing 110 and the upper housing 112 are shown, which together form the rigid housing that encloses and aligns the internal closure mechanism of the self-closing needle assembly. The lower housing 110 serves as the bottom half of the rigid housing and is constructed, for example, from a molded medical-grade thermoplastic to ensure durability, biocompatibility, and ease of manufacturing. The lower housing 110 encloses and supports internal components—including the tube spring 705 and the pliable tubing segment 703—when assembled with the upper housing 112, thereby providing structural integrity and precise alignment for reliable operation of the closure mechanism.

The lower housing 110 is configured with openings 114 positioned and sized to receive pins 715 of a security tape assembly 102, enabling the pins to pass into the housing and engage the tube spring 705 to hold its jaws 1207, 1208 open during normal operation. The alignment of the openings 114 with the internal tube spring 705 ensures that the pins 715 can reliably maintain patency of the pliable tubing segment 703. Upon withdrawal of the pins 715, the tube spring 705 is released, allowing the jaws 1207 and 1208 to close and compress the tubing segment 703 to halt fluid flow.

A valley 1002 is formed within the lower housing 110 and is shaped to securely seat and support the lower jaw portion of the tube spring 705, thereby maintaining positional stability and alignment of the spring relative to the pliable tubing segment 703. The valley 1002 provides a stable seating surface that distributes forces exerted by the spring and prevents deformation or misalignment of the housing during use.

The upper housing 112 includes corresponding openings 1114 that cooperate with the openings 114 of the lower housing 110 to provide a continuous passage for the pins 715 into the tube spring’s jaw opening 1210. The upper housing 112 further includes a valley 1102 that mirrors the valley 1002 of the lower housing 110 and is contoured to receive the upper jaw portion of the tube spring 705. When the upper housing 112 and lower housing 110 are assembled, the valleys 1102 and 1002 together define a guided cavity that captures the tube spring 705 and positions it precisely with respect to the flexible tubing segment 703 passing through the spring’s end wall opening 1202.

The cooperative geometry of the openings 114 and 1114 ensures accurate alignment of the security pins 715 with the tube spring jaws 1207 and 1208 to maintain patency during use and to permit reliable withdrawal of the pins upon device separation from the adhered security tape assembly. The paired valleys 1002 and 1102 provide secure seating and directional control for the tube spring 705, preventing rotational or lateral displacement and promoting repeatable binary closure of the pliable tubing segment 703 when the pins 715 are withdrawn. The two housing parts 110, 112 are shaped to be joined together—for example by adhesive bonding, welding, or mechanical capture—forming a rigid enclosure that protects the spring-biased clip, aligns the closure mechanism, and guides the fluid pathway from the cannula side to the distal medical tubing connection 116.

Referring now to FIGS. 10B and 11B, a second example of the housing is shown comprising a lower housing 113 and an upper housing 115 that, when assembled, form a rigid enclosure for the internal closure mechanism of the self-closing needle assembly. In this embodiment, only the lower housing 113 includes openings 114. The openings 114 are positioned and sized to accommodate pins 715 of a security tape assembly, allowing the pins 715 to pass into the housing and engage the tube spring to hold its jaws open during normal operation. The lower housing 113 further includes a groove 1003 contoured to securely seat and align the tube spring, providing positional stability and guiding the spring with respect to the pliable tubing segment.

The upper housing 115 is formed with a flat bottom edge configured to mate with the lower housing 113. The upper housing 115 includes an internal groove 1103 that mirrors the corresponding seating features of the lower housing and provides complementary support for the tube spring when the housings are joined. Because the upper housing 115 does not include pin openings, the flat bottom edge mates with the lower housing 113 in a manner that leaves the openings 114 of the lower housing unobstructed after assembly. As a result, when the two housing parts 113 and 115 are combined, the openings 114 remain open to receive the pins 715, ensuring that the pins can be inserted and withdrawn reliably to maintain patency or permit closure of the tubing segment.

This second housing configuration simplifies alignment of the pins 715 by providing a single set of openings 114 in the lower housing 113 while the upper housing 115 supplies mating structure and spring seating without obstructing pin access. The cooperative geometry of the groove 1003 in the lower housing 113 and the groove 1103 in the upper housing 115 stabilizes the tube spring and prevents rotational or lateral displacement. When the pins 715 are withdrawn from the openings 114, the tube spring closes to compress the pliable tubing segment and halt fluid flow. The two housing parts 113 and 115 are joinable by adhesive bonding, welding, or mechanical capture to form a rigid enclosure that protects the spring-biased clip and maintains an unobstructed pathway for pin engagement through the openings 114.

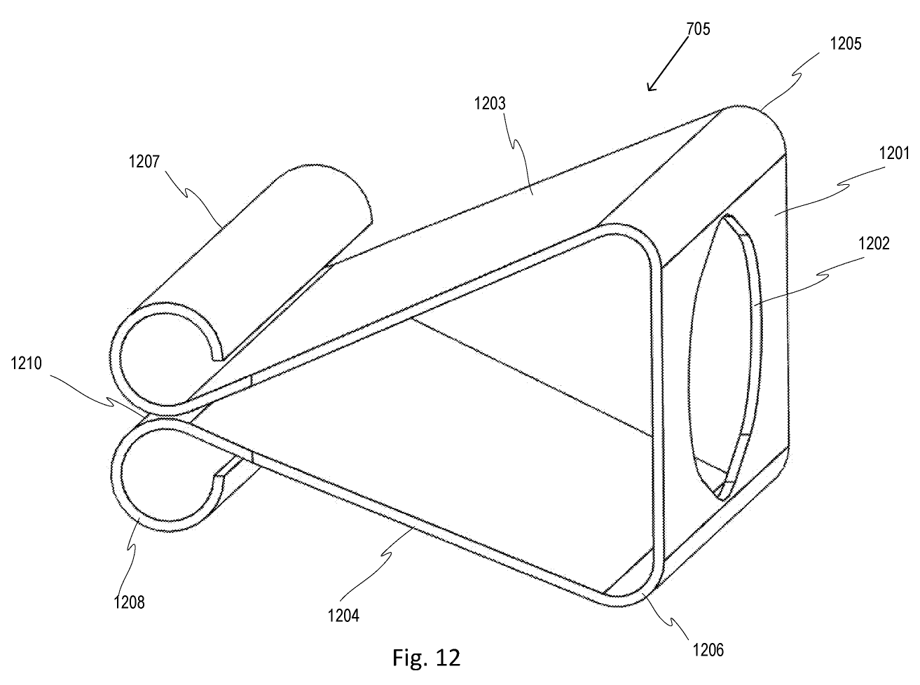

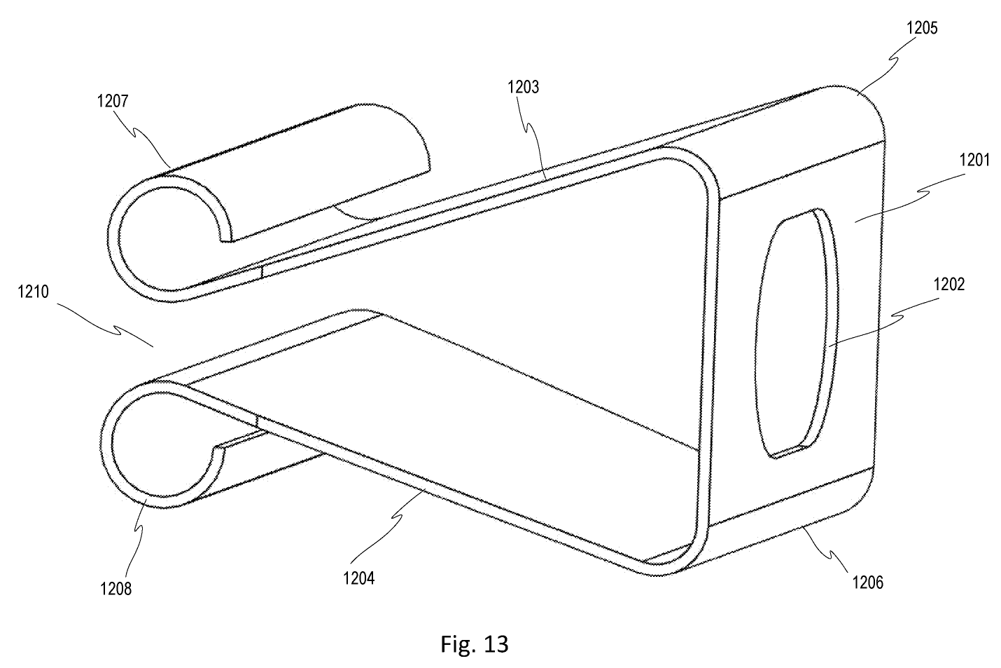

Referring now to FIGS. 12 and 13, a tube spring 705 is shown in two operating conditions. FIG. 12 illustrates the tube spring 705 in its naturally closed position, and FIG. 13 illustrates the tube spring 705 in an opened position. The tube spring 705 is biased toward the closed condition and will not remain open without external force applied by pins 715; the pins 715 are omitted from FIGS. 12 and 13 for clarity. In embodiments, the tube spring 705 may be made of a metal, such as spring steel. In other embodiments, the tube spring 705 may be made out of a polymer having a specified flexibility that makes it capable of remining open by pins 715, and closing if the pins 715 are withdrawn from the jaw opening 1210.

Other embodiment are contemplated where the jaw opening 1210 is formed not from two jaws of the tube spring 705, but rather by other mechanical structure, including two magnets configured to pull toward each other through magnetic force, linear springs arranged opposed walls of the rigid housing, and inflated bladders of pressurized gas similarly mounted and exerting force on the tube passing through the jaw opening or equivalent opening between these alternative force generating mechanisms.

The tube spring 705 includes an end wall 1201 having an opening 1202 sized to receive a pliable tubing segment. The end wall 1201 extends between a top corner 1205 and a bottom corner 1206 that provide structural continuity to a top wall 1203 and a bottom wall 1204. From the corners 1205 and 1206, the top wall 1203 and the bottom wall 1204 extend forward and converge toward a jaw region. In the illustrated embodiment, the walls 1203 and 1204 with the end wall 1201 define a substantially triangular profile, but the walls may also have a curved shape.

At the distal ends of the walls 1203 and 1204, respective jaw structures are formed. An upper jaw 1207 and a lower jaw 1208 are shown as rolled or curved segments that define a jaw opening 1210. In alternative embodiments, the jaws may be flat and set at an angle to facilitate insertion of pins 715. Thus, the opening of the jaws facing the needle has a tapered profile to facilitate the insertion of pins 715. The pins 715 themselves may have tapered ends, or be tapered along their entirety or their portion, to make insertion of the pins into the jaws easier.

In the closed state of FIG. 12, the inherent spring bias draws the upper jaw 1207 and lower jaw 1208 together so that the jaw opening 1210 collapses and compresses a pliable tubing segment positioned between the jaws, thereby halting fluid flow. In the opened state of FIG. 13, external force provided by pins 715 (not shown for clarity) spreads the jaws 1207 and 1208 apart to form the jaw opening 1210, maintaining patency of the tubing segment that passes through the jaw opening 1210 and the end wall opening 1202.

The end wall opening 1202 guides the tubing segment through the tube spring 705, and the converging top and bottom walls 1203 and 1204 direct force to the jaws 1207 and 1208. During normal operation, pins 715 enter the jaw opening 1210 to hold the jaws open; when the pins 715 are withdrawn, the spring bias returns the tube spring 705 to the closed condition shown in FIG. 12, compressing the tubing segment to stop fluid flow.

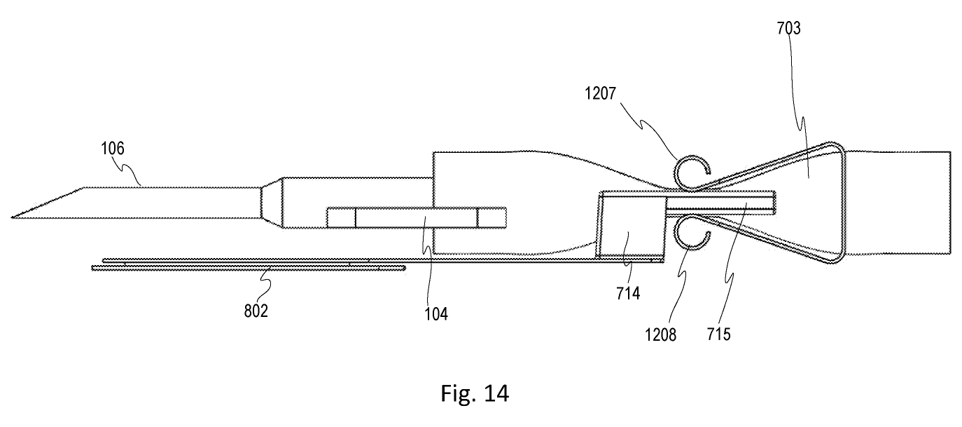

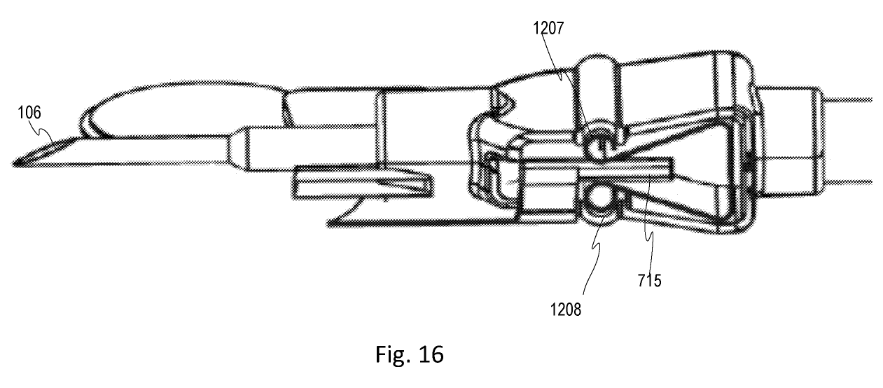

Referring now to FIGS. 14 and 16, a cutaway side view of a self-closing needle assembly is shown in which pins 715 are inserted into a jaw opening 1210 of a tube spring 705 to hold the spring in an open position. FIG. 16. illustrates the rigid housing interacting with the tube spring 705, with upper jaw 1207 seated in valley 1102 and the lower jaw 1208 seated in groove 1103. In FIG. 14, the housing is removed for increased clarity and to emphasize the interaction of the spring 705 and the pliable tubing segment 703. In the illustrated configuration, a needle 106 protrudes from a needle barrel 108 having wings 104 that provide lateral stabilization when taped to a patient. A security tape assembly includes a skin contact patch 802 positioned beneath the cannula region for adhesion to the patient’s skin, and a pillar 714 that supports pins 715 extending generally parallel to the planar support of the security tape assembly.

The tube spring 705 is positioned within the housing region adjacent the needle barrel 108. The tube spring 705 includes an upper jaw 1207 and a lower jaw 1208 that, in the absence of external force, converge under spring bias to close the jaw opening 1210. In FIG. 14, the pins 715 are shown inserted between the upper jaw 1207 and lower jaw 1208, spreading the jaws apart and maintaining the jaw opening 1210 in the open condition. With the jaws held open, a pliable tubing segment 703 passes through the tube spring 705 without being compressed, thereby maintaining patency for fluid flow.

The cutaway view emphasizes the cooperative interaction among the pins 715, the tube spring 705, and the pliable tubing segment 703. While the pins 715 remain engaged within the jaw opening 1210, the tube spring 705 is prevented from closing and fluid can pass through the pliable tubing 703. Upon withdrawal of the pins 715—such as when the needle assembly separates from the adhered security tape assembly—the spring bias of the tube spring 705 drives the upper jaw 1207 and lower jaw 1208 together, collapsing the jaw opening 1210 to compress the pliable tubing segment 703 and stop fluid flow.

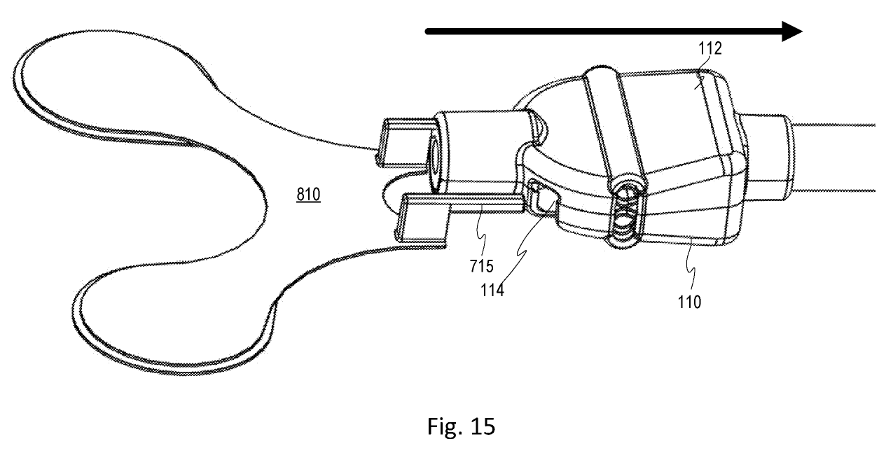

Referring now to FIG. 15, a side view illustrates withdrawal of pins 715 from openings 114 in the rigid housing formed by lower housing 110 and upper housing 112. A bold arrow indicates the direction of movement of the rigid housing while the security tape assembly 810 carrying the pins 715 remains in place. This results in the withdrawal of the pins 715 from the openings 114. As the pins 715 retract from the jaw opening 1210 of the tube spring 705, the spring bias drives the jaws closed, compressing the pliable tubing segment 703 and stopping fluid flow.

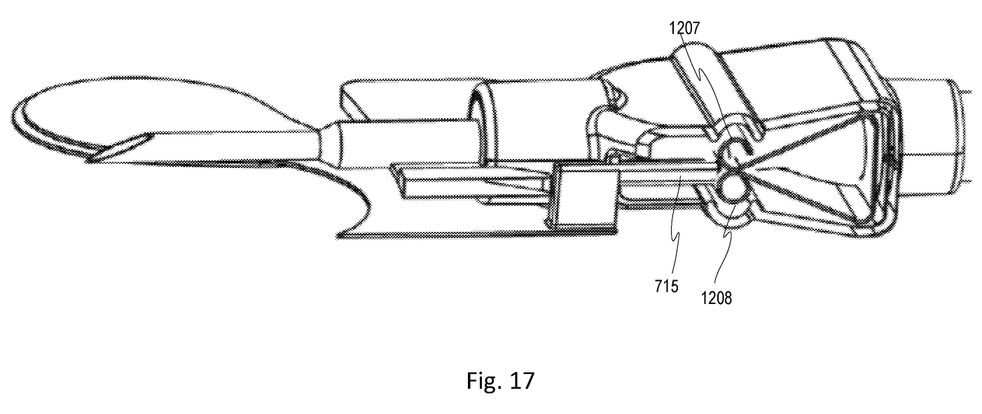

Referring now to FIG. 17, a cutaway side view of the self-closing needle assembly is shown in a condition following withdrawal of pins 715 from the jaw opening 1210 of the tube spring 705. In this state, the inherent spring bias draws the upper jaw 1207 and lower jaw 1208 together so that the jaw opening 1210 collapses. As the jaws close, they pinch and compress the pliable tubing segment 703 positioned between them, thereby occluding the lumen of the tubing and stopping fluid flow through the assembly. This figure illustrates the automatic shut-off action that occurs when the pins 715 are removed—such as during separation of the rigid housing from the adhered security tape assembly—demonstrating the binary transition from a held-open, patent condition to complete closure of the tubing segment 703.

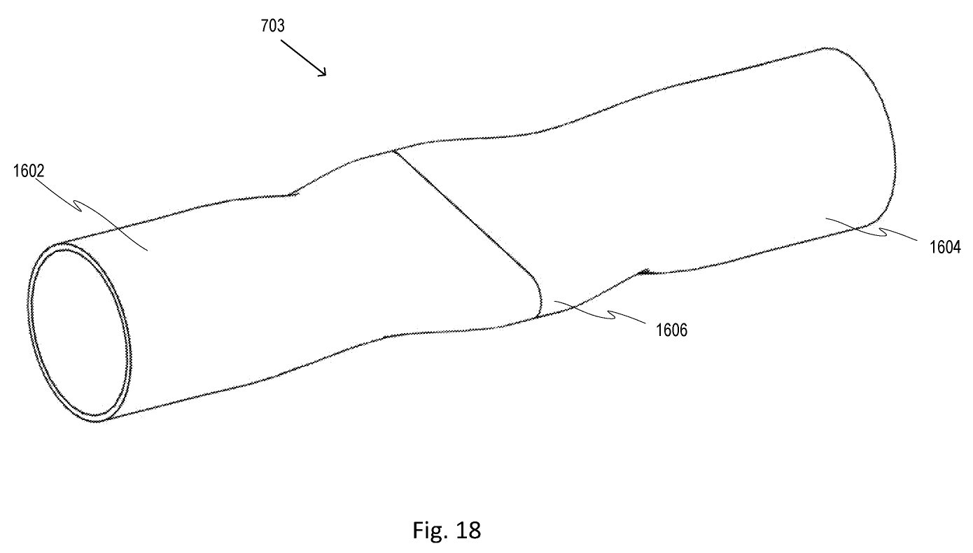

Referring now to FIG. 18, a pliable tubing segment 703 is shown. In the illustrated embodiment, the tubing segment 703 includes two rounded portions 1602 and 1604 and a molded flattened portion 1606 positioned between the rounded portions. The rounded portions 1602 and 1604 provide generally circular cross-sections suitable for connection to adjacent components, such as a support tube segment at one end and medical tubing at the other end. The flattened portion 1606 is configured to be positioned between the jaws of the tube spring so that, when the jaws close, the flattened portion 1606 is reliably compressed to occlude flow. This geometry preserves lumen patency in the rounded end regions while presenting a predictable, easily occluded section in the middle to enable repeatable binary shut-off when the spring-biased clip closes.

In embodiments, the pliable tubing segment 703 is formed from elastomeric materials such as silicone, latex, thermoplastic elastomer (TPE), or polyurethane. To promote predictable occlusion under spring force, the material hardness can be selected within a Shore A durometer range of about 20A to 60A, more preferably 25A to 45A, and in some examples about 30A to 40A. Wall thickness in the flattened portion 1606 may be reduced relative to the rounded portions 1602, 1604, for example to a nominal thickness of about 0.30 mm to 0.80 mm, more preferably 0.40 mm to 0.60 mm, while the rounded portions may have a wall thickness of about 0.50 mm to 1.20 mm. Suitable outside diameters for the rounded portions 1602, 1604 can be about 2.0 mm to 4.0 mm, more preferably 2.5 mm to 3.5 mm, with corresponding inside diameters yielding a lumen adequate for intended flow rates.

The flattened portion 1606 can present a compressed width when occluded of less than about 0.10 mm under the spring force, and is configured so that the spring closes the lumen fully at applied jaw forces of about 2 N to 8 N, more preferably 3 N to 6 N. These quantitative characteristics ensure that the tubing is sufficiently pliable to be pinched closed reliably by the tube spring while maintaining structural integrity and kink resistance at connection regions.

In some embodiments, the pliable tubing segment 703 is optional. The medical tubing 116 itself may be sufficiently pliable to pass through the end wall opening 1202 and the jaw opening 1210 of the tube spring 705, enabling the jaws 1207, 1208 to compress the medical tubing 116 directly to occlude flow when the pins 715 are withdrawn. Utilizing the medical tubing 116 as the pliable segment (which can be easily pinched closed) can simplify assembly by reducing the number of distinct components, eliminate interfaces between different tubing materials, and lower manufacturing costs while preserving the binary shut-off performance of the self-closing needle assembly.

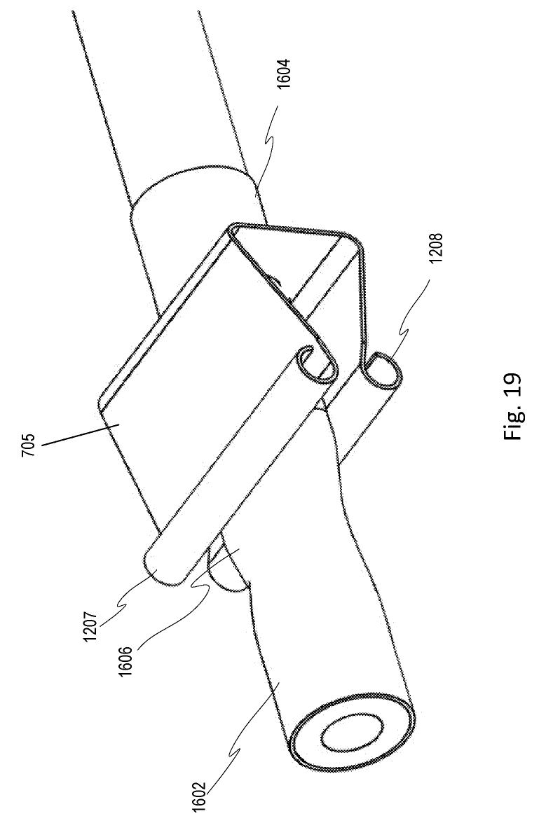

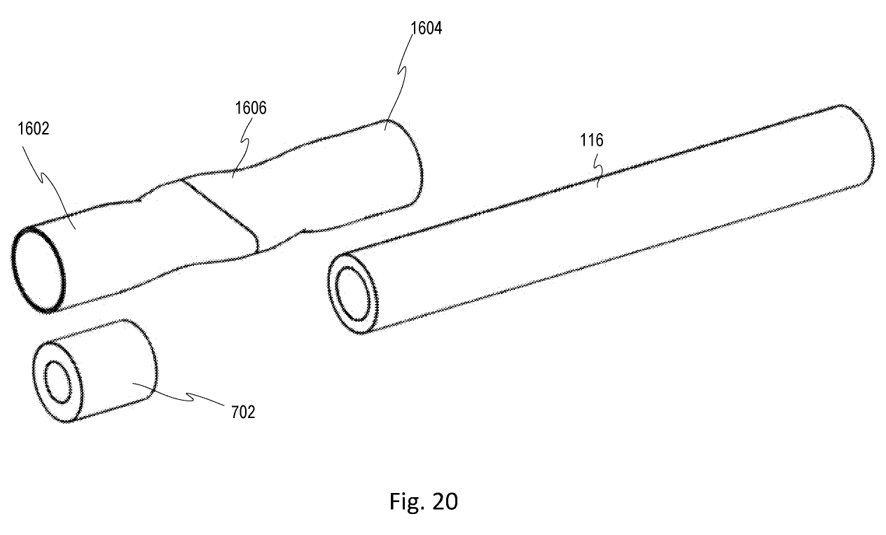

Referring now to FIGS. 19 and 20, details of the interaction between a tube spring 705 and a pliable tubing segment 703, as well as an example construction of the flow path incorporating the tubing segment 703, are shown. In FIG. 19, the pliable tubing segment 703 includes two rounded portions 1602 and 1604 separated by a molded flattened portion 1606. The flattened portion 1606 is positioned between an upper jaw 1207 and a lower jaw 1208 of the tube spring 705. When the jaws are held open by pins (not shown in FIG. 19), the tubing segment 703 remains patent; when the pins withdraw and the tube spring 705 closes, the jaws 1207, 1208 pinch the flattened portion 1606 to occlude the lumen and stop flow.

FIG. 20 illustrates a possible construction of the flow path that integrates the pliable tubing segment 703 with adjacent components. Because the pliable tubing segment 703 may be more compliant than medical tubing 116, one or more support tube segments 702 can be inserted into one or both rounded ends 1602, 1604 of the tubing segment 703. The support tube segment 702 provides local reinforcement to resist collapse, preserve lumen shape, and facilitate secure attachment to neighboring components such as medical tubing 116, a needle barrel 108, and, in some embodiments, an extension tube 109. In assembly, the reinforced end 1602 can couple to the cannula side (e.g., needle barrel 108 or extension tube 109), while the opposite end 1604 can couple to medical tubing 116 leading distally to external systems. The use of support tube segment(s) 702 enables reliable transitions between materials of differing pliability, maintains kink resistance at junctions, and preserves predictable occlusion behavior at the flattened portion 1606 within the tube spring 705.

It is, thus, apparent that there is provided, in accordance with the present disclosure, a self-closing needle and fluid loss prevention methods. Many alternatives, modifications, and variations are enabled by the present disclosure. Features of the disclosed embodiments can be combined, rearranged, omitted, etc., within the scope of the invention to produce additional embodiments. Furthermore, certain features may sometimes be used to advantage without a corresponding use of other features. Accordingly, Applicants intend to embrace all such alternatives, modifications, equivalents, and variations that are within the spirit and scope of the present invention.

1. A self-closing needle assembly comprising:
a housing having a front tube opening and a rear tube opening;
a spring-biased clip disposed within the housing and having opposing jaws configured to compress a tubing segment that is positioned between the opposing jaws;
the tubing segment positioned in the housing and passing through the front tube opening and the rear tube opening, and fluidly connected a needle configured to be inserted into a surface; and
a security tape assembly configured to adhere to said surface and configured to hold the opposing jaws of the spring-biased clip open while the housing is positioned in a predetermined position relative to the security tape assembly, wherein
displacement of the housing from the predetermined position relative to the security tape assembly causes the security tape assembly to release the opposing jaws of the spring-biased clip to close and thereby pinch the tubing segment closed.

2. The self-closing needle assembly according to claim 1, further comprising:
a hub attached to the needle; and
a pair of wings extending from the hub.

3. The self-closing needle assembly of claim 1, wherein the spring-biased clip comprises a tube spring biased toward a closed position.

4. The self-closing needle assembly of claim 1, wherein the housing includes openings, and
the security tape assembly includes pins sized to fit through said openings and to hold the jaws open.

5. The self-closing needle assembly of claim 4, wherein the security tape assembly includes
a flexible planar support carrying skin-contact adhesive patches configured to adhere the flexible planar support to the surface into which the needle is inserted; and
the pins are a pair of pins carried by the flexible planar support and arranged to extend into the openings of the housing.

6. The self-closing needle assembly of claim 5, wherein the security tape assembly further includes
pillars extending perpendicularly from the flexible planar support, the pins being mounted on the pillars.

7. The self-closing needle assembly of claim 1, wherein the spring-biased clip includes an end wall having an aperture through which the tubing segment passes.

8. The self-closing needle assembly of claim 2, wherein the tubing segment includes a flattened portion positioned between the jaws for reliable occlusion by the spring-biased clip.

9. The self-closing needle assembly of claim 8, wherein the flattened portion includes a flexible tubing segment that further includes a support tube segment inserted into an end of the flexible tubing segment to provide structural support at a connection to the hub.

10. The self-closing needle assembly of claim 1, wherein
the tubing segment extends out of the housing and toward the needle, defining an extension tubing segment.

11. The self-closing needle assembly of claim 10, wherein
the extension tubing segment has a length between 1 and 2 times the length of the needle.

12. A method for preventing fluid loss during needle disconnection, comprising:
providing a needle assembly comprising a housing, a spring-biased clip within the housing, and a tubing segment extending between jaws of the spring-biased clip to a needle;
providing a security tape assembly that holds open the jaws of the spring-biased clip to maintain patency a fluid path through the tubing segment;
holding the jaws of the spring-biased clip open by the security tape assembly;
inserting the needle into a surface;
adhering the security tape assembly to said surface to thereby maintain the housing in a predetermined position relative to the security tape assembly; and
in response to withdrawal of the needle from said surface while the security tape assembly remains adhered, permitting the jaws to close and compress the tubing segment to stop fluid flow through the tubing segment.

13. The method of claim 12, wherein the holding the jaws of the spring-biased clip open includes inserting a pair of pins of the security tape assembly through openings in the housing and between the jaws.

14. The method of claim 12, wherein adhering the security tape assembly comprises exposing an adhesive by removing a cover from contact patches on a planar support and pressing the contact patches to the surface.

15. The method of claim 13, wherein the permitting the jaws to close the tubing segment is binary such that the tubing segment is either fully open while the jaws are held open by the pins or fully closed after the pins are withdrawn from the housing.

16. A method of securing a needle assembly, the method comprising:
providing the needle assembly comprising a rigid housing, a spring clip disposed within the housing, and a tubing segment passing through the spring clip;
wherein the spring clip comprises an end wall having a hole, a top wall having a first jaw, and a bottom wall having a second jaw, the walls forming a substantially triangular shape;
wherein the tubing segment passes through the hole and between the first and second jaws;
providing a security tape assembly comprising a planar support structure and two pins extending parallel to the planar support structure;
inserting the two pins through openings in the rigid housing and between the first and second jaws to maintain the first and second jaws in an open position;
adhering the security tape assembly to a patient; and
wherein upon withdrawal of the needle assembly from the patient while the security tape assembly remains adhered to the patient, the two pins withdraw from between the first and second jaws, allowing the first and second jaws to close and compress the tubing segment to block fluid flow through the tubing segment.
